(19) United States
(12) Patent Application Publication
(10) Pub. No.: US 2006/0174898 A1
(43) Pub. Date: Aug. 10, 2006

(54) Defibrillator insertion device and method

(75) Inventor: Ward M. Brown, La Crosse, WI (US)

Correspondence Address: OPPENHEIMER WOLFF & DONNELLY LLP, 45 SOUTH SEVENTH STREET, SUITE 3300, MINNEAPOLIS, MN, 55402, US

(73) Assignee: Team Brown Enterprises, LLC, La Crosse, WI (US)

(21) Appl. No.: 11/054,802

(22) Filed: Feb. 10, 2005

(51) Int. Cl. A61B 19/00

(52) U.S. Cl. 128/899

(57) ABSTRACT

A device is provided that may be used to implant a subcutaneous device such as an ICD or a pacemaker. The device includes a body defining a compartment for holding the ICD during insertion and a blade assembly that cuts a path toward the targeted implantation site. A handle assembly including a plunger is used to expel the ICD from the compartment once the targeted implantation site has been reached.

### BACKGROUND OF THE INVENTION

The device of the present invention relates to a device that can be used to simply and safely insert a subcutaneous device such as an implantable defibrillator. Such devices shall hereinafter be referred to broadly as ICDs (Implantable Cardiac Devices). ICDs are typically flat devices, about the size of soap bars, which are implanted in the chest on the patient's left side just up under the collarbone.

Typical implantation procedures involve using a local anesthetic at the insertion site and a narcotic known as a “twilight” sedative, which relaxes the patient. A first incision is made through the skin, and subsequent, deeper incisions are made in order to provide a pocket into which the device may be placed. These subsequent incisions are less precise than the initial incision and require a degree of experience and skill to prevent unnecessary trauma, as it is difficult to make the incisions only as large as necessary to accommodate the device.

Once the incisions are made, the attending physician pushes the device into the desired position with his or her fingers. While en route to the targeted implantation site, the device pushes away surrounding tissue. This insertion method places stress on the connection between the leads of the device and the device itself as the leads are being pulled by the tissue passing over the device.

It would be beneficial to provide a delivery or insertion device that could be used to place a subcutaneous device. It would also be beneficial to provide a delivery or insertion device that protects the subcutaneous device while en route to the targeted implantation site. It would be even more beneficial to provide an insertion device that includes a cutting implement that makes an optimally sized incision to create a pocket in a patient for accepting a subcutaneous device.

### BRIEF SUMMARY OF THE INVENTION

The present invention pertains to a delivery device that is useable to implant a subcutaneous device, such as an ICD, into a patient. The device is constructed and arranged to provide a protective vehicle for the ICD during the delivery procedure as well as a surgical tool used to make the precise tissue cuts necessary for the implantation procedure.

The device includes a body defining a compartment sized to receive an ICD. At a proximal end of the body is a handle useable to manipulate the device during an implantation procedure. The handle includes a plunger slideably disposed within the handle. A distal end of the plunger projects into the compartment. A proximal end of the plunger protrudes from a proximal end of the handle. The plunger is used to eject the ICD from the compartment and into the patient when the targeted location has been reached.

At a distal end of the device is a pair of pivoting blades. The blades move from a closed position to an open position. In the closed position, the blades form a sharp leading edge for the delivery device. The blades cut a path for the device as the device is being advanced. Once the physician has determined that the device is appropriately positioned, the physician pushes the plunger into the handle. The plunger ejects the ICD out of the distal end of the body. As the ICD is being pressed forward, it forces the blades apart, slicing an appropriately sized pocket through the patient's tissue for receiving the ICD.

Therefore, one aspect of the present invention is a delivery device for a subcutaneous device comprising a body, a blade assembly, a handle, and a plunger. The body defines a compartment for containing a subcutaneous device to be delivered. The blade assembly is operably attached to a distal side of the body. The handle is operably attached to a proximal side of the body. And the plunger is slideably attached to the handle and has a distal end that extends into the compartment. The plunger is movable from a first position whereby the distal end of the plunger is located in a proximal end of the compartment and a second position whereby the distal end of the plunger is located in a distal end of the compartment.

Another aspect of the invention is a method of implanting a subcutaneous device. The method begins with identifying an entry point on the patient and cleaning the entry point. Next the entry point is anesthetized and an incision is made. The delivery device of the present invention, loaded with the subcutaneous device, is inserted into the incision. Next, the delivery device is advanced by applying forward pressure on the handle, thereby causing the blade assembly to cut at least some of the surrounding tissue while the delivery device is being advanced. Once in place, the plunger is slid into the compartment of the delivery device, thereby ejecting the subcutaneous device from the compartment. Finally, the delivery device is withdrawn, leaving the subcutaneous device in the patient.

### BRIEF DESCRIPTION OF THE DRAWINGS

FIG. 1 is a plan view of the present invention;

FIG. 2 is a perspective view of a distal end of the present invention; and,

FIG. 3 is a plan view of an ICD being ejected from the present invention.

### DETAILED DESCRIPTION OF THE INVENTION

Referring now to the Figures, there is shown a delivery device 10 of the present invention. The delivery device 10 generally includes a body 12, a handle 14 at a proximal end of the body 12, and a blade assembly 14 at a distal end of the body 12.

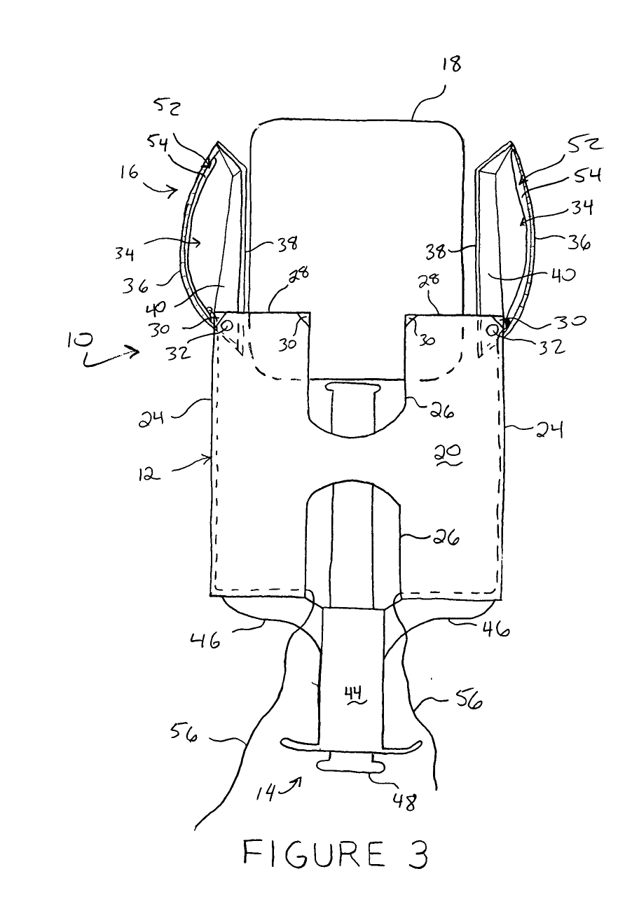

The body 12 defines a compartment that is sized to receive an ICD 18 (FIG. 3) and is more specifically defined by an upper wall 20, a lower wall 22 and a pair of opposed sidewalls 24. The upper and lower walls 20 and 22 are shown as including optional cutouts 26. The cutouts 26 facilitate easy cleaning, if the device 10 is to be constructed as a reusable instrument, and also prevent the introduction of an air bubble into the implantation site.

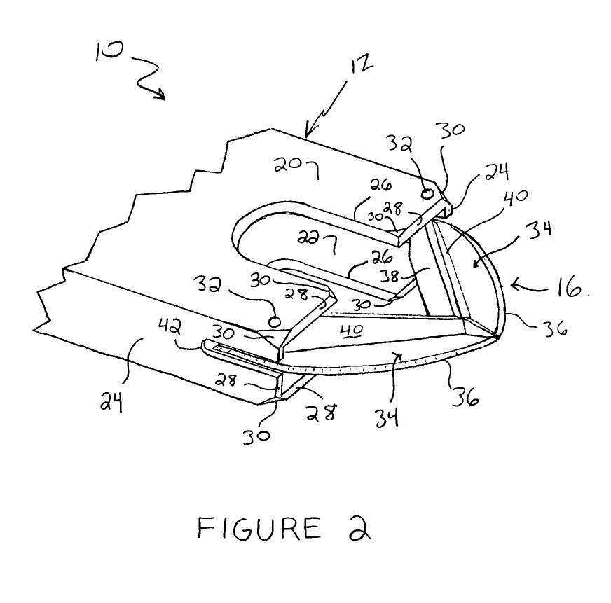

Distal edges 28 of the walls 20, 22, and 24 define a distal opening of the compartment. As shown in FIG. 2, the distal edges 28 include chamfered corners 30 to prevent trauma to surrounding tissue during insertion. It is also envisioned to round the distal edges 28. The body 12 also includes at its distal end, a pair of pivot pins 32 that pivotally attach the blade assembly 16 to the body 12.

The blade assembly 16 includes at least one, preferably two, blades 34. The blades 34 include sharpened distal edges 36 that act as scalpels during insertion. The blades 34 also include broad proximal edges 38 that provide a solid surface against which an ICD 18 can act when forcing the blades 34 apart. The broad proximal edges 38 also prevent damage from being imparted to the ICD 18. Between the sharpened distal edges 36 and the broad proximal edges 38, the each blade 34 preferably includes at least one angled surface 40 making a gradual transition from the sharpened distal edge 36 to the broad proximal edge 38 for the tissue to follow during insertion.

The blades 34 are pivotally attached to the body 12 with hinge pins 32. The blades are thus able to pivot from the closed position shown in FIG. 2, to the open position shown in FIG. 3. The blades 34 are shown as being curved, though they may be any shape. Preferably, in the closed position, the blades 34 generally form a wedge shape to facilitate insertion ease. The shape of the blades 34, and the location of the hinge pins 32, may require slots 42 to be formed in the sidewalls 24. The slots 42 provide room for the blades to travel to the open position. Alternatively, the blades 34 could be shaped such that the sharpened distal edges 36 recede to the broad proximal edge 38 distal of the distal edges 28 of the sidewalls 24.

The blades 34 may pivot freely around the hinge pins 32, relying on the resistance of the tissue to keep them in a closed position during insertion. Alternatively, a small spring may be operably attached to the blades 34, urging them closed until the spring force is overcome by ejecting an ICD 18 through the blades 34. This embodiment ensures that no additional cuts are made while removing the device. The spring force should be light enough to prevent the blades 34 from grasping the sides of an ICD 18 and pulling it during retraction of the device 10.

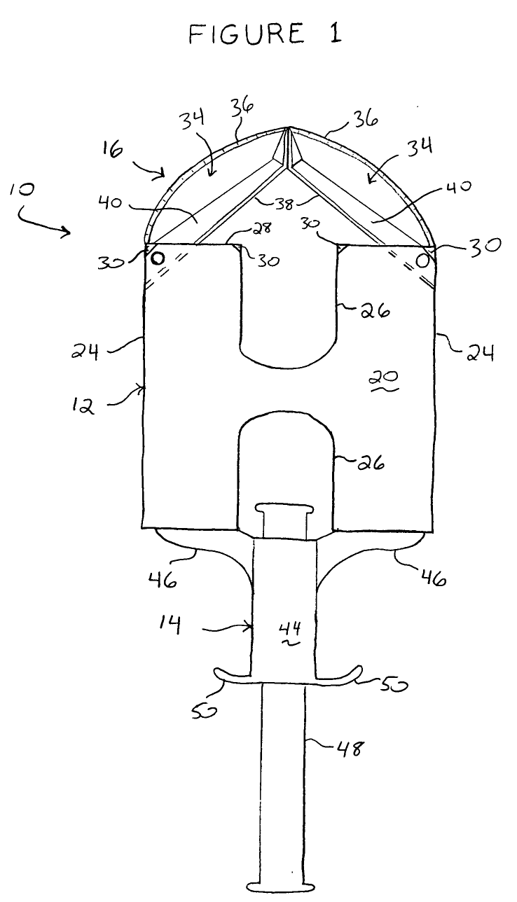

At the distal end of the body 12 is a handle assembly 14, shown in FIGS. 1 and 3. The handle assembly 14 includes a handle 44 operably attached to the body 12 with braces 46. The handle assembly 14 also includes a plunger 48 passing through the handle 44 and slideably disposed therein. The plunger 48 is moveable between a first, retracted position shown in FIG. 1, to a second, deployed position shown in FIG. 3. In order to provide one-handed operation, the handle assembly preferably includes finger grips 50 extending outwardly from the handle 44.

An optional feature, shown in FIG. 3, is a cauterizing mechanism 52. The cauterizing mechanism shown in FIG. 3 includes heating elements 54 operably disposed on or near the sharpened distal edges 36 of the blades 34. Power is provided to the heating elements 54 via leads 56 that run along the interior sides of the body 12 and out one of the cutouts 26 near the proximal end of the body 12. The proximal ends of the leads 56 are connected to a controllable power supply (not shown).

A method of using the device 10 to implant an ICD is also provided. An entry point is identified on the patient into which the device 10 is to be inserted. The entry point is then cleaned and draped in a usual fashion. Local anesthetic is infiltrated and an incision, preferably on the order of 0.5 to 1.0 cm is made utilizing a standard scalpel.

The insertion device 10 is loaded by placing an ICD 18 or other subcutaneous apparatus in the compartment of the body 12. Optimally, the device 10 is designed specifically for the apparatus to be delivered such that the size of the compartment of the body 12 closely matches the size of the apparatus.

With the blade assembly 16 in the closed position, the most distal portions of the sharpened edges 36 are placed in the previously made incision. Then, the physician applies forward pressure on the handle 44, advancing the insertion device 10 while causing the blade 34 to cut through the tissue between the incision and the targeted implantation site. If the device is equipped with a cauterizing mechanism 52, power is supplied to the mechanism 52 such that the incisions made by the blades 34 are instantly cauterized, thereby minimizing bleeding and the need for internal, post-implantation sutures.

Once the targeted location is reached, or more specifically, when the device 10 is in a position whereby the ICD will be in the targeted location once deployed, the forward pressure on the handle 44 is no longer applied.

Next, the physician depresses the plunger 48 while holding the handle 44 stationary relative to the patient. Doing so advances the ICD 18 by causing a distal end of the plunger to act against a proximal end of the ICD 18, forcing the ICD 18 out of the distal opening of the body 12. As the ICD 18 progresses distally, the distal end of the ICD 18 acts against the broad proximal edges 38 of the blades 34, causing the blades 34 to pivot around the hinge pins 30.

Once the blades 34 have reached a fully open position, shown in FIG. 3, the ICD 18 is expelled out of the body 12 and into the pocket created by the blades 34. The device 10 is then withdrawn from the patient leaving the ICD 18 in the targeted location.

The invention has herein been described in its preferred embodiments to provide those skilled in the art with the information needed to apply the novel principles and to construct and use the embodiments of the examples as required. However, it is to be understood that the invention can be carried out by specifically different devices and that various modifications can be accomplished without departing from the scope of the invention itself.

By way of example only, the blades could be modified to achieve a variety of different shapes, sizes, and angles. Also, the pivot points of the blades could be relocated. Blades could be provided above and/or below the exit path of the ICD such that the blades would not need to pivot out of the way of the ICD as it is plunged out of the compartment. The blades could be configured to excrete a coagulating chemical to limit bleeding and speed healing. The handle could be configured with a trigger mechanism that either operates the movement of the blades, the expulsion of the ICD, or both.

Thus, it is important to understand that any such modifications are considered within the scope of the invention, which is set out in the following claims:

What is claimed is:

1. A delivery device for a subcutaneous device comprising:
a body defining a compartment for containing a subcutaneous device to be delivered;
a blade assembly operably attached to a distal side of the body;
a handle operably attached to a proximal side of the body;
a plunger slideably attached to the handle, the plunger having a distal end extending into the compartment, the plunger movable from a first position whereby the distal end of the plunger is located in a proximal end of the compartment and a second position whereby the distal end of the plunger is located in a distal end of the compartment.

2. The delivery device of claim 1 wherein the body comprises an upper wall, a lower wall and a pair of side walls connecting the upper wall and the lower wall.

3. The delivery device of claim 2 wherein the body further comprises at least one cutout, defined by at least one of the walls, leading to the compartment, the cutout preventing air from becoming trapped in the compartment during an insertion procedure.

4. The delivery device of claim 2 wherein the upper wall, the lower wall, and the side wall each include a distal edge.

5. The delivery device of claim 4 wherein the distal edges comprise chamfered corners.

6. The delivery device of claim 1 wherein the blade assembly comprises at least one blade pivotally attached to the body.

7. The delivery device of claim 6 wherein the blade is pivotally attached to the body with pins.

8. The delivery device of claim 6 wherein the at least one blade comprises a pair of blades.

9. The delivery device of claim 6 wherein the at least one blade comprises a sharpened distal edge.

10. The delivery device of claim 9 wherein the at least one blade further comprises a broad proximal edge.

11. The delivery device of claim 10 wherein the at least one blade further comprises at least one angled surface between the sharpened distal edge of the blade and the broad proximal edge of the blade.

12. The delivery device of claim 9 wherein the sharpened distal edge is curved.

13. The delivery device of claim 1 further comprising a cauterizing mechanism operably attached to the blade assembly.

14. The delivery device of claim 13 wherein the cauterizing mechanism comprises a heating element operably disposed on the blade assembly.

15. A method of implanting a subcutaneous device comprising:
identifying an entry point on the patient;
cleaning the entry point;
anesthetizing at least the entry point;
making an incision at the entry point;
providing a delivery device including:
a body defining a compartment sized to house the subcutaneous device therein;
a blade assembly operably attached to a distal end of the body;
a handle operably attached to a proximal end of the body; and,
a plunger slideably disposed within the handle and extending into the compartment;
placing the subcutaneous device into the compartment of the delivery device;
inserting the delivery device into the incision;
advancing the delivery device by apply forward pressure on the handle, thereby causing the blade assembly to cut at least some of the surrounding tissue while the delivery device is being advanced;
sliding the plunger into the compartment thereby ejecting the subcutaneous device from the compartment; and,
withdrawing the delivery device, leaving the subcutaneous device in the patient.

16. The method of claim 15 wherein anesthetizing at least the entry point comprises locally anesthetizing the entry point.

17. The method of claim 15 wherein anesthetizing at least the entry point comprises generally anesthetizing the patient.

18. The method of claim 15 wherein making an incision at the entry point comprises making an incision on the order of 0.5 cm to 1.0 cm long at the entry point.

19. The method of claim 15 providing a delivery device comprises providing a delivery device further including a cauterizing mechanism operably attached to the blade assembly.

20. The method of claim 19 further comprising the step of cauterizing the surrounding tissue while advancing the delivery device.

21. A delivery device for use in placing a subcutaneous apparatus in a patient comprising:
a first means for transporting the subcutaneous apparatus to a desired implantation site within the patient;
a second means for cutting tissue en route to the desired implantation site; and,
a third means for ejecting the subcutaneous apparatus from the first means.

22. The delivery device of claim 21 wherein the first means comprises a body defining a compartment for containing a subcutaneous apparatus.

23. The delivery device of claim 22 wherein the body comprises an upper wall, a lower wall and a pair of side walls connecting the upper wall and the lower wall.

24. The delivery device of claim 23 wherein the body further comprises at least one cutout, defined by at least one of the walls, leading to the compartment, the cutout preventing air from becoming trapped in the compartment during an insertion procedure.

25. The delivery device of claim 23 wherein the upper wall, the lower wall, and the side wall each include a distal edge.

26. The delivery device of claim 25 wherein the distal edges comprise chamfered corners.

27. The delivery device of claim 21 wherein the second means comprises a blade assembly operably attached to the first means.

28. The delivery device of claim 27 wherein the blade assembly comprises at least one blade pivotally attached to the first means.

29. The delivery device of claim 28 wherein the at least one blade comprises a pair of blades.

30. The delivery device of claim 28 wherein the at least one blade comprises a sharpened distal edge.

31. The delivery device of claim 28 wherein the at least one blade comprises a broad proximal edge.

32. The delivery device of claim 30 wherein the sharpened distal edge is curved.

33. The delivery device of claim 21 wherein the third means for ejecting the subcutaneous apparatus from the first means comprises a plunger slideably related to the first means.

34. The delivery device of claim 21 further comprising a fourth means for cauterizing tissue.

35. The delivery device of claim 34 wherein the fourth means comprises a heating element operably disposed on the second means.
